(19) United States
(12) Patent Application Publication
(10) Pub. No.: US 2014/0267684 A1
(43) Pub. Date: Sep. 18, 2014

(54) SYSTEM AND METHOD FOR DETECTING CONTAMINATION IN FOOD USING HYPERSPECTRAL IMAGING

(71) Applicant: CHEMLMAGE CORPORATION, PITTSBURGH, PA (US)

(72) Inventors: Matthew NELSON, Harrison City, PA (US); Patrick TREADO, Pittsburgh, PA (US); Charles GARDNER, Gibsonia, PA (US); Andrew BASTA, Cranberry Township, PA (US)

(73) Assignee: CHEMLMAGE CORPORATION, PITTSBURGH, PA (US)

(21) Appl. No.: 14/215,489

(22) Filed: Mar. 17, 2014

Related U.S. Application Data
(60) Provisional application No. 61/799,225, filed on Mar. 15, 2013.

(51) Int. Cl. G06T 7/00; G01N 33/02; G01N 21/94

(52) U.S. Cl. CPC G06T 7/0004; G01N 21/94; G01N 33/02
USPC 348/89

(57) ABSTRACT

The present disclosure provides systems and methods for determining the presence of a contaminate in a food sample. Interacted photons from a food sample having a contaminate of interest are collected. The interacted photons are passed through a tunable filter to a hyperspectral detector that generates a hyperspectral image representative of the filtered interacted photons. The hyperspectral image is analyzed by comparing the hyperspectral image obtained from the food sample to known hyperspectral images to identify a contaminate in the food sample. The systems and methods disclosed herein provide an easy and non-destructive tool for identifying contaminates in a food sample.

### CROSS REFERENCE TO RELATED APPLICATION

This application claims benefit of and priority to U.S. Provisional Application Ser. No. 61/799,225 entitled “System and Method for Detecting Contamination in Food Using Hyperspectral Imaging” filed Mar. 15, 2013, the disclosure of which is incorporated by reference herein in its entirety.

### BACKGROUND

Mass food contamination is a reality that, if unnoticed, can have widespread and potentially fatal consequences. Even with modern food health and safety guidelines, standards, and regulations for preparation and distribution, major food contamination outbreaks continue to occur. Therefore, the contamination of perishable goods needs to be detected and the cause identified before having the opportunity to escalate into an epidemic that could have a devastating effect on many people.

Melamine is a common chemical that has recently have been added to animal feed in an attempt to increase the apparent protein content of the product. However, melamine can have adverse effects on animals ingesting it. As a result, a substantial portion of animal feed that has recently entered the consumer market has been contaminated. This resulted in a large number of deaths in animals receiving such animal feed. Current methods of detecting melamine and other contaminants are labor-intensive and time consuming. There exists a need for rapid, non-destructive, specific, low-cost, and routine systems and methods for assessing feed samples for the presence of melamine. Additionally, it would be helpful to be able to detect other contaminants such as cyanuric acid, ammeline, and ammelide, which may also be found in animal feed. These agents may be present in animal feeds due to their individual addition to the feed or as a result of melamine degradation.

Desirably, fast melamine screening would be beneficial if it featured minimal sample preparation (e.g., no extraction/centrifugation), routine analysis of a number of samples without reagents, minimal processing procedures, and ease of operation. Such systems and methods are increasingly important due to the potential public and animal health concerns. In addition, systems and methods are needed for melamine screening to prevent protein fraud.

Spectroscopic imaging combines digital imaging and molecular spectroscopy techniques, which can include Raman scattering, fluorescence, photoluminescence, ultraviolet, visible and infrared absorption spectroscopies. When applied to the chemical analysis of materials, spectroscopic imaging is commonly referred to as chemical imaging. Instruments for performing spectroscopic (i.e., chemical) imaging typically comprise an illumination source, an image gathering optics, a focal plane array, imaging detectors, and imaging spectrometers.

In general, when performing spectroscopic imaging, certain features of the imaging device are determined by the sample size. For instance, sample size can determine the choice of image gathering optic. For example, a microscope is typically employed for the analysis of sub-micron to millimeter spatial dimension samples. For larger objects, in the range of millimeter to meter dimensions, macro-lens optics are appropriate. For samples located within relatively inaccessible environments, flexible fiberscope or rigid borescopes can be employed. For very large scale objects, such as planetary objects, telescopes are appropriate image gathering optics.

For detection of images formed by the various optical systems, two-dimensional, imaging focal plane array (“FPA”) detectors are typically employed. The choice of FPA detector is governed by the spectroscopic technique employed to characterize the sample of interest. For example, silicon (“Si”) charge-coupled device (“CCD”) detectors or CMOS detectors are typically employed with visible wavelength fluorescence and Raman spectroscopic imaging systems, while indium gallium arsenide (“InGaAs”) FPA detectors are typically employed with near-infrared spectroscopic imaging systems. For some modalities, intensified charge-coupled devices (“ICCD”) may also be used.

Spectroscopic imaging of a sample is commonly implemented by one of two methods. First, point-source illumination can be used on a sample to measure the spectra at each point of the illuminated area. Second, spectra can be collected over the entire area encompassing a sample simultaneously using an electronically tunable optical imaging filter, such as, an acousto-optic tunable filter (AOTF), a multi-conjugate tunable filter (MCF), or a liquid crystal tunable filter (LCTF). Here, the organic material in such optical filters is actively aligned by applied voltages to produce the desired bandpass and transmission function. The spectra obtained for each pixel of an image forms a complex data set referred to as a hyperspectral image. Hyperspectral images may contain the intensity values at numerous wavelengths or the wavelength dependence of each pixel element in the image. Multivariate routines, such as chemometric techniques, may be used to convert spectra to classifications.

Spectroscopic devices operate over a range of wavelengths due to the operation ranges of the possible detectors or tunable filters. This enables analysis in the Ultraviolet (“UV”), visible (“VIS”), near infrared (“NIR”), short-wave infrared (“SWIM”), mid infrared (“MIR”) wavelengths, long wave infrared wavelengths (“LWIR”), and to some overlapping ranges.

There currently exists a need for a non-destructive, accurate and reliable tool for determining the presence of a contaminate in a food sample.

### SUMMARY

In an embodiment, a system for identifying a contaminate in a food sample may include a first collection optic configured to collect a plurality of interacted photons. Interacted photons are those photons that have interacted with the food sample. The system further includes a tunable filter configured to filter a first plurality of interacted photons collected from the first collection optic. The tunable filter is configured to filter the first plurality of interacted photons into a plurality of wavelengths to generate filtered interacted photons. In the system, a hyperspectral detector is configured to detect the filtered interacted photons and to generate a hyperspectral image of the filtered interacted photons. The system further includes a processor configured to analyze the hyperspectral image of the plurality of filtered photons by comparing the hyperspectral image of the plurality of filtered photons to a database of known hyperspectral images in order to identify the contaminate in the food sample.

In another embodiment, the system may include a second collection optic configured to collect a second plurality of interacted photons. In one embodiment, a RGB detector is configured to detect the second plurality of interacted photons and to generate a RGB image representation of the second plurality of interacted photons.

In another embodiment, the system may include an illumination source configured to provide photons that interact with a sample to generate interacted photons. In one embodiment, the system described herein may be housed in a portable or handheld device.

In an embodiment, a method for identifying a contaminate in a food sample is provided. The method includes collecting a plurality of interacted photons from the plurality of interacted photons that have interacted with the food sample. The method further provides directing a first plurality of interacted photons through a tunable filter to generate a plurality of filtered photons where the filter separates the photons into a plurality of wavelengths. The method further provides detecting the plurality of filtered photons with a hyperspectral detector where the hyperspectral detector generates a hyperspectral representation of the plurality of filtered photons. The method further includes analyzing the hyperspectral image of the plurality of filtered photons by comparing the hyperspectral image of filtered photons to a database of known hyperspectral images to identify the contaminate in the food sample.

### BRIEF DESCRIPTION OF THE DRAWINGS

FIG. 1A is a schematic illustration of an illustrative system for identifying a contaminate in a food sample according to an embodiment;

FIG. 1B is a schematic illustration of an illustrative portable system for identifying a contaminate in a food sample according to an embodiment;

FIG. 1C is a schematic illustration of an illustrative handheld system for identifying a contaminate in a food sample according to an embodiment;

FIG. 2 is a flow-chart illustrating an illustrative method for identifying a contaminate in a food sample according to an embodiment;

FIG. 3A illustrates PLSR model results in a PLS calibration coefficient determined over the range of wavelengths for melamine identification in wheat flour according to an embodiment;

FIG. 3B illustrates SEC and SEP values for samples of wheat flour containing melamine in calibration and prediction sets according to an embodiment; and

FIG. 4 illustrates image intensities corresponding to various concentrations of melamine in wheat flour according to an embodiment.

### DETAILED DESCRIPTION

Reference will now be made in detail to embodiments of the present disclosure, examples of which are illustrated in the accompanying drawings. Wherever possible, the same reference numbers will be used throughout the specification to refer to the same or like parts. “Contaminate” as used herein includes any material that is undesired in a food sample and may include, without limitation, chemicals, pathogens, bacteria, viruses, and the like.

FIGS. 1A, 1B, and 1C depict illustrative of a systems 100 for identifying a contaminate according to embodiments herein. In one embodiment of the present system, the system 100 is housed in a portable system 101 or handheld unit 102. FIG. 1B and FIG. 1C illustrate an example of a portable and a handheld unit, respectively, featuring the system 100. In another embodiment, the system 100 contemplates designs to accommodate other portable configurations, such as, for example, a design having objectives on movable arms and the like.

Referring now to FIG. 1A, the system 100 comprises a RGB optical subsystem 105. The RGB optical subsystem 105 includes a RGB collection optic 110b and a RGB detector 120b. In one embodiment, the RGB collection optic 110b is a RGB lens. The RGB collection optic 110b is configured to collect a plurality of interacted photons that have interacted with a food sample. As used herein, “interacted photons” comprise photons scattered by a food sample, photons absorbed by a food sample, photons reflected by a food sample, photons emitted by a food sample or any combination thereof. In one embodiment, the RGB detector 120b is a RGB camera. The RGB collection detector 120b is configured to detect the interacted photons that have been collected from the RGB collection optic 110b. In one embodiment, the RGB optical subsystem 105 generates a RGB image representative of a location on a food sample representative of the interacted photons collected from the RGB collection optic 110b.

In another embodiment, the system 100 comprises a hyperspectral subsystem 106. The hyperspectral subsystem 106 may include a collection optic 110a, a tunable filter 115 and a hyperspectral detector 120a. The hyperspectral detector, as used herein, may be configured to detect any wavelength as apparent to those of skill in the art in view of this disclosure. In one embodiment, the hyperspectral detector may be configured to detect wavelengths from about 180 nm to about 2,500 nm. In another embodiment, the hyperspectral detector may be configured to detect wavelengths from about 380 nm to about 2,500 nm. In another embodiment, the hyperspectral detector may be configured to detect wavelengths from about 700 nm to about 2,500 nm. In yet another embodiment, the hyperspectral detector may be configured to detect wavelengths from about 850 nm to about 1,800 nm. In yet another embodiment, the hyperspectral detector may be configured to detect wavelengths from about 400 nm to about 1,100 nm. In yet another embodiment, the hyperspectral detector may be configured to detect wavelengths from about 1,000 nm to about 1,700 nm. It is understood that the hyperspectral detector can be configured to detect wavelengths in any subset of wavelengths within those disclosed herein based on a subset of wavelengths that may be of particular interest. In one embodiment, the collection optic 110a is a hyperspectral lens. The collection optic 110a is configured to collect a plurality of interacted photons that have interacted with the food sample. The tunable filter 115 is configured in a sequential manner with the collection optic 110a to filter photons collected from the collection optic. In another embodiment, the hyperspectral detector 120a is sequentially configured with the tunable filter to detect photons filtered by the tunable filter. The hyperspectral detector 120a, upon detection of the filtered photons, generates a hyperspectral image representative of the filtered photons. The hyperspectral image provides detailed imaging information to a user and may provide any of discrimination, identification, and concentration of materials of interest.

In one embodiment, the system 100 generates the RGB image and the hyperspectral image substantially simultaneously or contemporaneously. That is, the system 100 can operate to generate a RGB image while at the same time the system can generate a hyperspectral image without the need for consecutively detecting the RGB image and the hyperspectral image.

The system 100 can be used to determine the presence and, if desired, the concentration of a contaminate in a food sample. Applications where the system 100 would be suitable for providing identification of a contaminate in a food sample include, for example, applications where it is desired to identify a contaminate in a food sample in order to prevent such contaminates from entering a feed supply. The system can be used in feed supplies that are to be used by animals or humans as well as final packaged food products. A “Food sample,” as used herein, can include any food or feed intended for consumption or any staple or commodity used in the process of preparing a consumable product, such as, grains, meats, vegetables, and the like. Other suitable applications for the system disclosed herein would be apparent to those of skill in the art in view of this disclosure. Identification of a contaminate in a food sample, as used herein, may include detecting the contaminate, identifying the contaminate, classifying the contaminate, measuring the concentration of the contaminate or any combination thereof.

In one embodiment of the system, the tunable filter 115 is configured to filter a plurality of interacted photons into a plurality of wavelength bands. In another embodiment, the tunable filter 115 may comprise a liquid crystal tunable filter, a multi-conjugate tunable filter, an acousto-optical tunable filters, a Lyot liquid crystal tunable filter, a Evans Split-Element liquid crystal tunable filter, a Solc liquid crystal tunable filter, a Ferroelectric liquid crystal tunable filter, a Fabry Perot liquid crystal tunable filter, or any combination thereof.

In one embodiment of the present system 100, the hyperspectral detector 120a features a focal plane array. In another embodiment of the present system, the hyperspectral detector 120a may comprise a detector including, for example, a InGaAs detector, a CMOS detector, an InSb detector, a MCT detector, an ICCD detector, a CCD detector, or any combination thereof.

The system 100 further comprises an field programmable gate array (“FPGA”) 125 and/or other interface logic that is in communication with the hyperspectral detector 120a. In another embodiment, the FPGA 125 is in communication with the RGB detector 120b. The FPGA 125 may further include a FPGA memory source 130. The FPGA 125 may further be in communication with an application processor 135. In one embodiment, the application processor 135 is, for example, a CPU, a digital signal processor, or a combination thereof. The application processor 135 may further be in communication with interface features or peripherals, such as, for example, a user input 140, such as input buttons, an external interface 145, such as a USB, a user display 150, such as a LCD panel display, storage memory 155, such as an SD card, application memory 160, and other peripherals as would be apparent to those of skill in the art in view of this disclosure. In one embodiment of the system 100, the FPGA 125, application processor 135, memory source 130, storage memory 155, and application memory 160 are configured to operate the system 100 to analyze and store collected data and store reference data. In one embodiment, the system 100 comprises a reference database having a plurality of reference data sets where each reference data set is associated with a known material. Each reference data set may comprise a hyperspectral image of a known material such that the hyperspectral image obtained from the food sample and contaminate via the system 100 can be compared to each reference data set to identify the food sample and the contaminate, thereby, identifying the contaminate in the food sample. In one embodiment, the system is configured to measure and compare the hyperspectral images of the food sample and the contaminate to identify the concentration of the contaminate in the food sample. Once the identification of the food sample and the contaminate are obtained by the system 100, the result of the identification can be reported to a user through the display 150. The system 100 may also comprise a battery pack 165 for supplying power to the system 100.

The system 100 can be configured to operate at various distances from the collection optic 110a and the RGB collection optic 110b to the food sample. The operating distance is dependent on the specifications of the collection optic 110a and the RGB collection optic 110b and can be at least about 0.5 m or greater. In one embodiment, the operating range of the system 100 is at least about 0.5 m or greater. In another embodiment, the operating range of the system 100 is at least about 5 m or greater. In yet another embodiment, the operating range of the system 100 is from about 1 m to about 20 m. In another embodiment, the operating range of the system 100 is from about 0.5 m to about 10 m. It is apparent to one of skill in the art that the operating range of the system can be configured to operate in any range within those recited. Further, in one embodiment, the system 100 is capable of operating with adjustable optics such that the operating range of the system 100 can be adjusted without the need to modify the collection optic 110a and the RGB collection optic 110b. In another embodiment, the collection optics may be configured to change the Field of View (“FOV”) with regard to the sample. Configuring the FOV can be accomplished by, in a fixed collection optics system, by changing the collection optics to achieve the desired FOV or, in an adjustable collection optic system, by adjusting the collection optic to achieved the desired FOV. The desired FOV would be apparent to those of skill in the art in view of this disclosure. The system 100 can further include other optical devices such as, for example, additional lens, other image gathering optics, arrays, mirrors, beam splitters and the like. Additional elements suitable for use with the system 100 are apparent to those of skill in the art in view of this disclosure.

The system 100 can further be configured to generate hyperspectral images of a food sample having a contaminate in near real time. In one embodiment, the system 100 tracks a food sample generating up to 2 frames/second to allow for near real time analysis of a food sample.

In one embodiment, the system 100 includes an illumination source. The illumination source can be one illumination source or a plurality of illumination sources. The illumination source can be ambient light or light provided to the food sample from an active source working in conjunction with the system 100. In one embodiment, the illumination source illuminates the sample from a variety of different angles. An active illumination source when used with the system 100 enables the system to operate in low or variable light conditions. Any illumination sources suitable for use with the system 100 can be used and such illumination sources would be apparent to those of skill in the art in view of this disclosure.

FIG. 1B illustrates an illustrative portable system 101 for identifying a contaminate in a food sample according to an embodiment. The portable system 101 features a hyperspectral lens 110a and a RGB lens 110b in close proximity to allow for the collection of photons from a food sample for analyzing a RGB image and a hyperspectral image in one step. The hyperspectral lens 110a collects photons from a food sample and directs the photons through a liquid crystal tunable filter (“LCTF”) 115. The photons from the LCTF 115 then pass through a focusing lens 118 which focus the photons before passing the photons on to the hyperspectral camera 120a. The hyperspectral camera 120a detects the photons passing from the focusing lens 118 and generates a hyperspectral image representative of the photons. A processor 135 in communication with the hyperspectral camera 120a analyzes the hyperspectral image to identify a contaminate in a food sample. The portable system 101 further includes a RGB lens 110b and a RGB camera 120b where the RGB camera is configured to detect photons collected from the RGB lens. The RGB camera 120b generates a RGB image representative of the photons collected from the RGB lens 110b. The RGB camera 120b is further in communication with the processor 135 for analyzing the RGB image. The portable system includes user interface controls 140 to permit the user to interact with the portable system 101. Further, the portable system 101 includes a display 150 for displaying information obtained by the portable system to a user. The portable system 101 further includes a power source 165 for operating the portable system remotely.

FIG. 1C depicts an illustrative handheld system 102 to permit a user to carry the system for identifying a contaminate in a food sample according to an embodiment. The handheld system 102 includes a handle 117 for being carried by a user. The handheld system 102 further includes active illumination sources 180 for illuminating a food sample to generate photons that interact with a food sample. The active illumination sources 180 enable the handheld system 102 to operate in remote locations having inadequate illumination. The handheld system 102 includes a hyperspectral collection lens aperture 106 and a RGB collection lens aperture 105 for collecting photons generated by a food sample. The handheld system 102 further includes a display 150 for conveying data obtained by the handheld system 102 to a user. In operation, the handheld system 102 operates in similar fashion to the system 100, as described herein.

FIG. 2 depicts a flow diagram of an illustrative method 200 for identifying a contaminate in a food sample according to an embodiment. The method 200 may comprise collecting 210 a plurality of interacted photons from the food sample comprising a contaminate. These interacted photons may be generated by illuminating the food sample using an active illumination, a passive illumination, or any combination thereof. The interacted photons may comprise photons scattered by the food sample, photons reflected by the food sample, photons absorbed by the food sample, photons emitted by the food sample, or any combination thereof.

In one embodiment of the method 200, the interacted photons may be passed through a tunable filter. The tunable filter is configured to filter the interacted photons into a plurality of wavelength bands. A hyperspectral image may be generated 220 representative of the food sample comprising contaminate. The hyperspectral image may be analyzed 230. In one embodiment, the hyperspectral image is analyzed 230 by comparing the hyperspectral image of the food sample and the hyperspectral image of the contaminate to a reference data set where the reference data set includes known hyperspectral images to identify the contaminate in the food sample. In one embodiment, the comparison is accomplished by applying one or more chemometric techniques. Chemometric techniques suitable for use in the method include: principle components analysis, partial least squares discriminate analysis, cosine correlation analysis, Euclidian distance analysis, k-means clustering, multivariate curve resolution, band t. entropy method, mahalanobis distance, adaptive subspace detector, spectral mixture resolution, and Bayesian fusion. It is also contemplated that more than one chemometric technique may be applied. It is further contemplated that any chemometric method as known to those of skill in the art may be applied. In one particular embodiment, the chemometric technique comprises Partial Least Squares Regression (“PLSR”). PLSR provides a supervised classification technique that is a least-squares regression analysis variant. Supervised classification is a mathematical model building technique that establishes a relationship between a set of independent variables, such as, for example, hyperspectral spectra, and a dependent variable, such as, for example, contaminate concentration, based on a set of food samples for which the hyperspectral spectra are measured and the dependent variable concentrations are known. Known concentrations may be measured by complementary techniques as known to those of skill in the art. In one embodiment, PLSR provides a mathematical technique used to develop the dependent variable concentration model. Upon validation, the model can be used to calculate the dependent variable concentration for food samples that have not been included in the model development. That is, the validation can be extrapolated to provide information, i.e., concentration data, for unknown samples based on the known concentrations determined during the validation.

In one embodiment, the analysis may detect a contaminate in a food sample, associate the contaminate in the food sample with a known material, detect a difference between the contaminate and the food sample, detect more than one contaminate in the food sample, measure the concentration of the contaminate in the food sample, or any combination thereof.

### EXAMPLES

### Example 1

FIG. 3A and FIG. 3B provides an illustrative example of identifying melamine in wheat flour according to an embodiment. In this example, the Condor™ NIR chemical imaging device was used to identify melamine in wheat flour. The Condor™ is commercially available from Chemimage Corporation located in Pittsburgh, Pa. The system was set to operate at wavelengths ranging from 1,000 nm to 1,700 nm. In FIG. 3A, PLSR model results in a PLS calibration coefficient determined over the range of wavelengths are illustrated. The calculation of percent melamine for a given wheat sample containing melamine was calculated by multiplying the spectrum obtained in FIG. 3A by a regression coefficient vector using dot product calculation to build a calibration set of data. The standard error of calibration (SEC) was calculated for the calibration set of data using a root mean square error calculation. The PLSR model was validated by testing the model on a set of data that was not used in the model building but for which the melamine concentrations were measured by a complementary technique. This set of data was used to calculate the standard error of prediction (SEP). The SEC and SEP values for each sample in the calibration and prediction sets are shown in FIG. 3B. FIG. 4 illustrates the PLSR concentration image for the calibration and validation set obtained for the samples. Each pixel in the input image represents a spectrum containing a known concentration of melamine and is multiplied by the PLSR coefficient vector to generate the PLSR concentration image. As shown in FIG. 4, the image intensities correspond to the known concentrations. Thus, the concentration of melamine in the flour for unknown samples was determinable.

What is claimed is:

1. A system for identifying a contaminate in a food sample, the system comprising:
a first collection optic configured to collect a plurality of interacted photons that have interacted with the food sample;
a tunable filter configured to filter a first plurality of interacted photons collected from the first collection optic into a plurality of wavelengths to generate filtered interacted photons;
a hyperspectral detector configured to detect the filtered interacted photons and generate a hyperspectral image of the filtered interacted photons; and
a processor configured to analyze the hyperspectral image of the filtered interacted photons by comparing the hyperspectral image of the filtered interacted photons to a known hyperspectral image in order to identify the contaminate.

2. The system of claim 1, further comprising:
a second collection optic configured to collect a second plurality of interacted photons; and
a RGB detector configured to detect the second plurality of interacted photons collected from the second collection optic and generate a RGB image representation of the second plurality of interacted photons.

3. The system of claim 2, wherein the hyperspectral image of filtered interacted photons and the RGB image are generated simultaneously.

4. The system of claim 1, further comprising an illumination source wherein the illumination source is configured to provide photons that interact with the food sample to generate the plurality of interacted photons.

5. The system of claim 1, wherein the tunable filter comprises a liquid crystal tunable filter, a multi-conjugate tunable filter, an acousto-optical tunable filter, a Lyot liquid crystal tunable filter, an Evans Split-Element liquid crystal tunable filter, a Solc liquid crystal tunable filter, a Ferroelectric liquid crystal tunable filter, a Fabry Perot liquid crystal tunable filter, or any combination thereof.

6. The system of claim 1, wherein the hyperspectral detector comprises an InGaAs detector, a CMOS detector, an InSb detector, a MCT detector, an ICCD detector, a CCD detector, or any combination thereof.

7. The system of claim 1, wherein the hyperspectral detector comprises a focal plane array.

8. The system of claim 1, further comprising a display configured to display hyperspectral analysis information obtained by the system to a user.

9. The system of claim 1, further comprising a user interface configured receive one or more inputs from a user of the system.

10. The system of claim 1, wherein the processor is further configured to analyze the hyperspectral image by applying a chemometric technique.

11. The system of claim 10, wherein the chemometric technique comprises principle components analysis, partial least squares discriminate analysis, cosine correlation analysis, Euclidian distance analysis, k-means clustering, multivariate curve resolution, band t. entropy method, mahalanobis distance, adaptive subspace detector, spectral mixture resolution, Bayesian fusion, or any combination thereof.

12. The system of claim 1, wherein the system is housed in a portable or handheld unit.

13. The system of claim 1, wherein the processor is further configured to determine the concentration of the contaminate in the food sample.

14. The system of claim 1, wherein the hyperspectral detector is configured to detect wavelengths from about 850 nm to about 1,800 nm.

15. The system of claim 1, wherein the hyperspectral detector is configured to detect wavelengths from about 700 nm to about 2,500 nm.

16. A method for identifying a contaminate in a food sample, the method comprising:
collecting a plurality of interacted photons from the food sample, wherein the plurality of interacted photons have interacted with the food sample;
directing a first plurality of interacted photons through a filter to generate a plurality of filtered photons, wherein the filter separates the first plurality of interacted photons into a plurality of wavelengths;
detecting the plurality of filtered photons with a hyperspectral detector,
generating a hyperspectral image of the plurality of filtered photons; and
analyzing the hyperspectral image of the plurality of filtered photons by comparing the hyperspectral image of the plurality of filtered photons to a database of known hyperspectral images to identify the contaminate.

17. The method of claim 16, further comprising:
collecting a second plurality of interacted photons; and
detecting the second plurality of interacted photons with a RGB detector, wherein the RGB detector generates a RGB image of the second plurality of interacted photons.

18. The method of claim 17, wherein the hyperspectral image of the plurality of interacted photons and the RGB image are generated simultaneously.

19. The method of claim 17, further comprising illuminating the food sample with an illumination source, wherein the illumination source provides photons that interact with the food sample to generate the second plurality of interacted photons.

20. The method of claim 16, further comprising illuminating the food sample with an illumination source wherein the illumination source provides photons that interact with the sample to generate the first plurality of interacted photons.

21. The method of claim 16, wherein analyzing the hyperspectral image further comprises applying a chemometric technique.

22. The method of claim 16, wherein analyzing further comprises determining the concentration of the contaminate in the food sample.

23. The method of claim 16, wherein the hyperspectral detector is further configured to detect wavelengths from about 850 nm to about 1,800.

24. A system for identifying a contaminate in a food sample, the system comprising:
an illumination source configured to provide photons that interact with the food sample;
a first collection optic configured to collect a first plurality of interacted photons where the first plurality of interacted photons includes photons that have interacted with the food sample;
a second collection optic configured to collect a second plurality of interacted photons where the second plurality of interacted photons includes photons that have interacted with the food sample;
a tunable filter configured to filter the first plurality of interacted photons collected from the first collection optic into a plurality of wavelengths to generate filtered interacted photons;
a hyperspectral detector configured to detect the filtered interacted photons, wherein the hyperspectral detector generates a hyperspectral image of the filtered interacted photons;
a RGB detector configured to detect the second plurality of interacted photons wherein the RGB detector generates a RGB image of the second plurality of interacted photons; and
a processor configured to analyze the hyperspectral image of the filtered interacted photons and compare the hyperspectral image of the filtered interacted photons to a database of known hyperspectral images in order to identify the chemical composition of the contaminate in the food sample.

25. The system of claim 24, wherein the hyperspectral image of the filtered interacted photons and the RGB image are generated simultaneously.

26. The system of claim 24, wherein the tunable filter comprises a liquid crystal tunable filter, a multi-conjugate tunable filter, an acousto-optical tunable filter, a Lyot liquid crystal tunable filter, an Evans Split-Element liquid crystal tunable filter, a Solc liquid crystal tunable filter, a Ferroelectric liquid crystal tunable filter, a Fabry Perot liquid crystal tunable filter, or any combination thereof.

27. The system of claim 24, wherein the hyperspectral detector comprises a InGaAs detector, a CMOS detector, an InSb detector, a MCT detector, an ICCD detector, a CCD detector, or any combination thereof.

28. The system of claim 24, wherein the hyperspectral detector comprises a focal plane array.

29. The system of claim 24, further comprising a display configured to display hyperspectral analysis information obtained by the system to a user.

30. The system of claim 24, further comprising a user interface configured receive one or more inputs from a user of the system.

31. The system of claim 24, wherein the processor is further configured to analyze the hyperspectral image of the filtered interacted photons by applying a chemometric technique.

32. The system of claim 31, wherein the chemometric technique comprises principle components analysis, partial least squares discriminate analysis, cosine correlation analysis, Euclidian distance analysis, k-means clustering, multivariate curve resolution, band t. entropy method, mahalanobis distance, adaptive subspace detector, spectral mixture resolution, Bayesian fusion, or any combination thereof.

33. The system of claim 24, wherein the system is housed in a portable or handheld unit.

34. The system of claim 24, wherein the processor is further configured to measure the concentration of the contaminate in the food sample.

35. The system of claim 24, wherein the hyperspectral detector is configured to detect wavelengths from about 850 nm to about 1,800.

36. The system of claim 24, wherein the hyperspectral detector is configured to detect wavelengths from about 700 nm to about 2,500 nm.
